(19) United States
(12) Patent Application Publication
(10) Pub. No.: US 2008/0294056 A1
(43) Pub. Date: Nov. 27, 2008

(54) BISPECTRAL PEROPERATIVE OPTICAL PROBE

(75) Inventors: Jerome Boutet, Grenoble (FR); Philippe Peltie, Saint Paul De Varces (FR)

Correspondence Address: LOWE HAUPTMAN & BERNER, LLP, 1700 DIAGONAL ROAD, SUITE 300, ALEXANDRIA, VA, 22314, US

(73) Assignee: COMMISSARIAT A L'ENERGIE ATOMIQUE, PARIS (FR)

(21) Appl. No.: 12/126,828

(22) Filed: May 23, 2008

(30) Foreign Application Priority Data
May 25, 2007 (FR) 07 03738

(51) Int. Cl. A61B 5/00

(52) U.S. Cl. 600/476

(57) ABSTRACT

The general field of the invention is that of peroperative optical probes designed to assist surgeons in performing medical procedures. The optical probes of the fluorescence type according to the invention are designed to be used on living tissues where the diseased areas have been marked by a fluorescent marker. They have dual lighting. The first situated in the red or near-infrared spectrum is necessary for achieving the fluorescence of the marked areas and for obtaining an image exploitable by a camera. The second situated in the visible spectrum is necessary for illuminating the marked areas with visible light, thus making the surgeon's work easier. The visible lighting can be punctiform or can be provided by an image projector. In the latter case, the projected image illuminates only the diseased areas.

### RELATED APPLICATIONS

The present application is based on, and claims priority from, French Application Number 07 03738, filed May 25, 2007, the disclosure of which is hereby incorporated by reference herein in its entirety.

1. Field of the Invention

The field of the invention is that of peroperative optical probes designed to assist surgeons in performing medical procedures.

2. Description of the Prior Art

Surgeons use optical probes that can be positioned over a patient. The optical probes function with visible light in order to illuminate the biological tissues on which the surgeons are operating. These probes are notably used for detection and elimination of certain tumours. These optical probes act as a microscope, and they are traditionally connected to a computer for displaying the biological tissues illuminated by the probe. Using a simple enlarged image of the biological tissues, the surgeon's experience is critical in determining the difference between healthy tissue and diseased tissue. When the visible differences are not sufficiently apparent, the surgeon performs a biopsy of the areas he considers suspect. If this biopsy proves positive, the surgeon removes a layer of tissues, then removes more cells, carries out another biopsy and recommences the operation until the biopsy is negative. This operation takes time and sometimes remains incomplete, since some affected areas are invisible to the naked eye or are insufficiently differentiated to be recognized by the surgeon.

To improve these devices, and to make it easier to detect diseased areas, it is possible to use fluorescent markers. These markers are used in the field of genetic and immunological analyses in vitro. The general principle entails marking a molecule, called probe, with a fluorescent marker. In the presence of the target molecule, this probe binds to the latter. The fluorescence is measured by exciting the marker with the aid of a light beam having a suitable wavelength. Following this excitation, the fluorophore relaxes, emitting a light at a wavelength greater than the excitation length. By means of specific filters, it is possible to detect, locate and quantify this fluorescence and thereby determine the presence of the target molecule.

The application of fluorescence to in vivo imaging is fairly recent. This is because living tissues absorb the visible wavelengths generally emitted by fluorescence. However, recent advances have allowed this technique to be applied to living tissues. These advances mainly concern:
The development of light sources towards wavelengths situated in the red or near-infrared spectrum. These wavelengths pass through the biological tissues better, with haemoglobin being the main light absorber. It is preferable for the wavelengths to be not greater than 900 nanometers so as to avoid absorption by water;
The increased power of the sources of the lighting systems. These sources are in most cases light-emitting diodes or laser diodes;
The improved sensitivity of the photodetectors.

Thus, in vivo fluorescence imaging systems have been developed. They are intended more especially for small laboratory animals, such as mice, for monitoring the development of tumours or for studying the efficacy of medicaments.

In the field of human medicine, prototype probes using fluorescence have been developed. These probes comprise essentially a laser and a camera. It is possible, for example, for the camera to be arranged over the operating table. In this case, the visual field is very substantial and does not allow high resolutions to be obtained. It is also possible to use probes that are sufficiently small to be held in the hand. In this case, the resolution is much greater. Thus, a probe having a field of the order of 8 centimetres and a working distance of the order of 10 to 15 centimetres has a resolution of less than 100 microns.

In all cases, the image supplied by the camera is sent back to a monitor, which can be a computer screen. The surgeon thus sees a magnified fluorescence image of the area illuminated by the probe. A device of this type is described in French patent FR 2 882 147 entitled “Dispositif d'imagerie de fluorescence par réflexion à deux longeurs d'onde” [Fluorescence imaging device with two-wavelength reflection]. This patent describes a device comprising an additional improvement. The source of excitation comprises two excitation wavelengths, making it possible to overcome the autofluorescence of the living tissues.

However, these probes of the fluorescence type have some disadvantages. Thus, the surgeon has to constantly glance from his screen to the operating site. Moreover, his surgical instruments do not appear in fluorescent light. Consequently, he cannot get his surgical manoeuvre to coincide precisely with the diseased cells he wishes to remove.

### SUMMARY OF THE INVENTION

The object of the invention is to overcome the above disadvantages. To this end, the optical probes of the fluorescence type according to the invention have dual lighting. The first is necessary for achieving the fluorescence of the marked areas and for obtaining an image exploitable by a camera. The second is necessary for illuminating or spotlighting the marked areas with visible light, while leaving the unmarked areas in shadow, thus making the surgeon's work easier.

The following advantages are thereby achieved:
The surgeon sees the diseased areas directly under visible light;
The viewing comfort for the surgeon is increased. He no longer needs to look back and forth between his computer screen and the patient's body;
The precision of the surgical manoeuvre is improved;
The duration of the surgical intervention is reduced.

More precisely, the invention relates to a peroperative optical probe comprising at least:
a first light source emitting a first light beam intended to illuminate a biological tissue surface, of which at least one area, the marked area, has previously been fixed by a fluorescent marker, said beam being emitted at a first wavelength corresponding substantially to the wavelength of excitation of the marking fluorophore, said fluorophore emitting at a second wavelength;
a first optical device comprising a first lens and a photosensitive surface, said lens forming, on said photosensitive surface, a first image of the biological tissue surface, the first device being connected to means for processing the image issuing from the photosensitive surface; characterized in that said probe also comprises:
optical means that are different from the first optical device and that are designed to illuminate at least a part of the biological tissue surface.

Advantageously, this surface part illuminated by the optical means corresponds to at least one of the marked areas identified by the means for processing the image.

Advantageously, in a first embodiment, the optical means comprise at least one image projector which, at the command of a control device analyzing the signal issuing from the processing means, generates a second image at at least a third wavelength situated in the visible spectrum, said second image being representative of the marked area, of which second image a second lens forms a third image on the biological tissue surface, the second device being arranged in such a way that this third visible image is of the same dimensions as and in coincidence with at least a marked area of the biological tissue surface.

In this case, the optical means can comprise a splitter allowing the second wavelength to be divided spectrally from the third wavelength, said splitter being arranged in such a way that the parts of the optical axes of the first lens and of the second lens that are situated between the biological tissue surface and said splitter are common.

Advantageously, the first optical device comprises a first optical filter transparent to the second wavelength, and the image projector comprises a second optical filter transparent to the third wavelength.

Advantageously, in a second embodiment, the optical means comprise at least a third light source emitting a third beam at the first wavelength and arranged in such a way as to illuminate the centre of the biological tissue surface in a quasi-punctiform manner.

In this case, the optical means can comprise a fourth light source emitting a fourth beam at a fourth wavelength situated in the visible spectrum, and arranged in such a way as to illuminate the centre of the biological tissue surface in a quasi-punctiform manner in coincidence with the second beam.

Advantageously, the optical means comprise a splitter allowing the second wavelength to be divided spectrally from the fourth wavelength, said splitter being arranged in such a way that the parts of the optical axes of the first lens and of the fourth source that are situated between the biological tissue surface and said splitter are common.

Advantageously, the optical means comprise an image generator which, from the signal issuing from the processing means, generates a second image at at least a third wavelength situated in the visible spectrum, said second image being representative of the marked area, of which second image a second lens forms a third image on the biological tissue surface, the optical means being arranged in such a way that this third visible image is of the same dimensions as and in coincidence with the biological tissue surface.

Advantageously, the image-processing means comprise means for identifying the marked areas, and said probe comprises electronic means for activating the fourth light source when a marked area is illuminated by the third light beam.

The invention relates more generally to a medical apparatus designed to be connected to a probe having all or some of the above means, and comprising said processing means and the device for control of the fourth light source.

Advantageously, the medical apparatus comprises a display device for viewing the equivalent of the first image or of the second image and comprises lighting of the scialytic type that emits at wavelengths different from at least the first wavelength.

Still other objects and advantages of the present invention will become readily apparent to those skilled in the art from the following detailed description, wherein the preferred embodiments of the invention are shown and described, simply by way of illustration of the best mode contemplated of carrying out the invention. As will be realized, the invention is capable of other and different embodiments, and its several details are capable of modifications in various obvious aspects, all without departing from the invention. Accordingly, the drawings and description thereof are to be regarded as illustrative in nature, and not as restrictive.

### BRIEF DESCRIPTION OF THE DRAWINGS

The present invention is illustrated by way of example, and not by limitation, in the figures of the accompanying drawings, wherein elements having the same reference numeral designations represent like elements throughout and wherein:

FIG. 1 shows a first embodiment of a probe according to the invention.

FIG. 2 shows a variant of this first embodiment.

FIG. 3 shows a second embodiment of a probe according to the invention.

FIG. 4 shows a variant of this second embodiment.

### DETAILED DESCRIPTION OF THE INVENTION

The following conventions have been adopted in the various figures. The single arrowheads in thin lines represent the direction of the light rays, and the dot-and-dash lines represent the optical axes of the lenses.

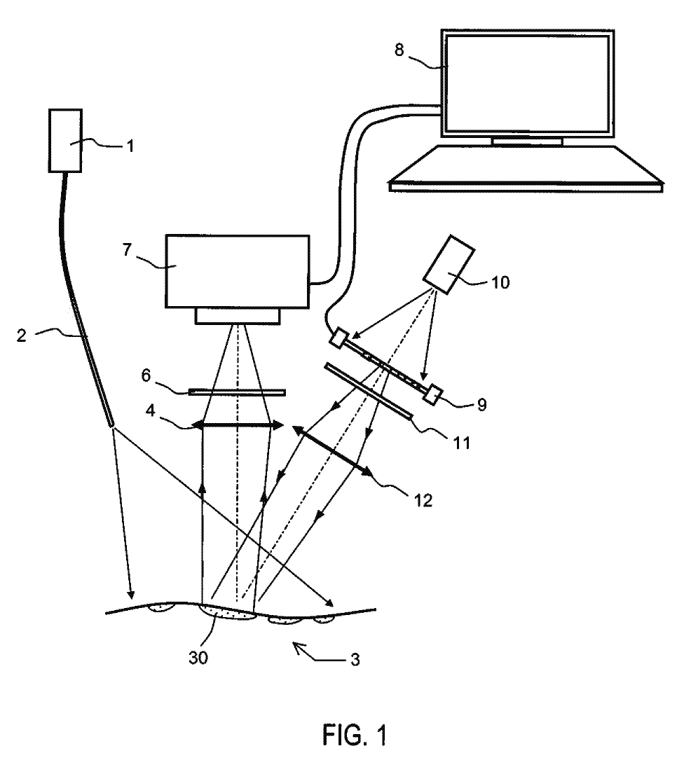

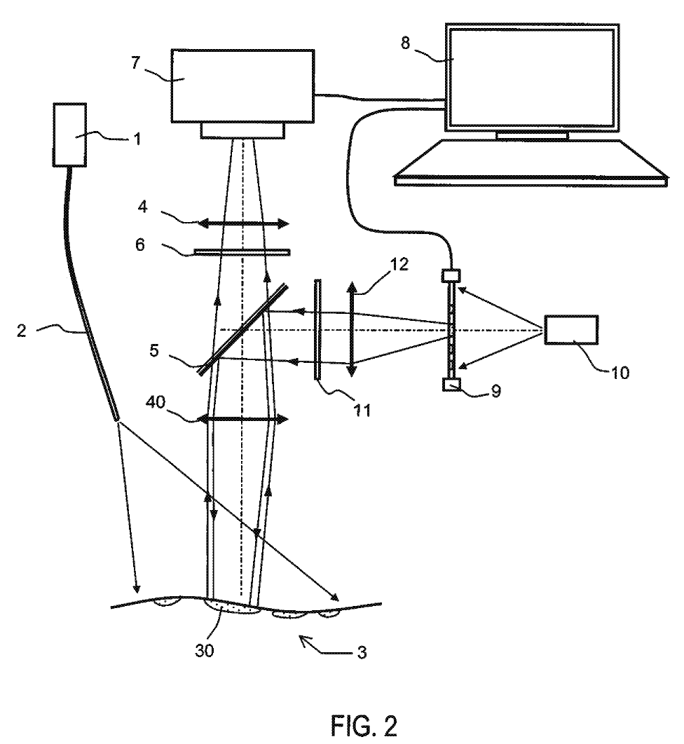

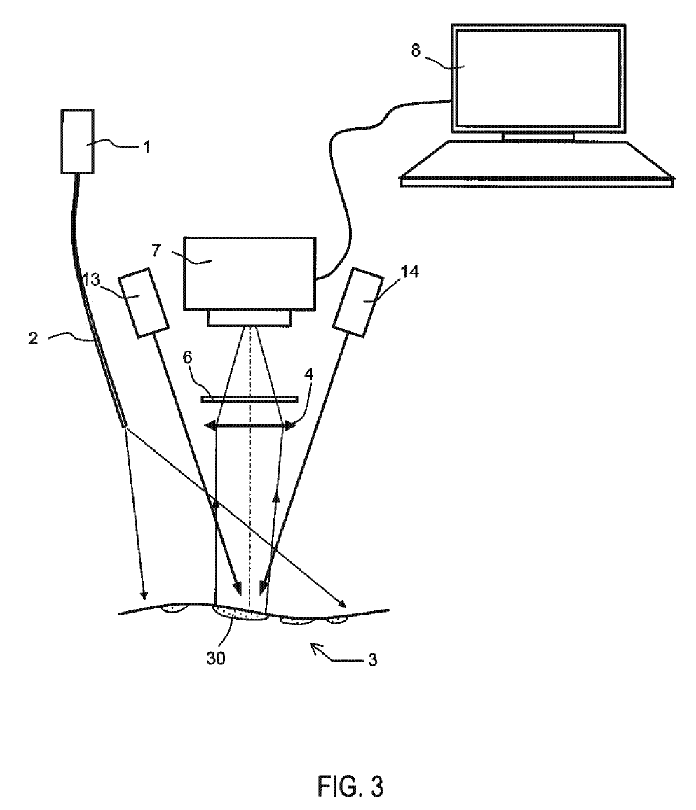

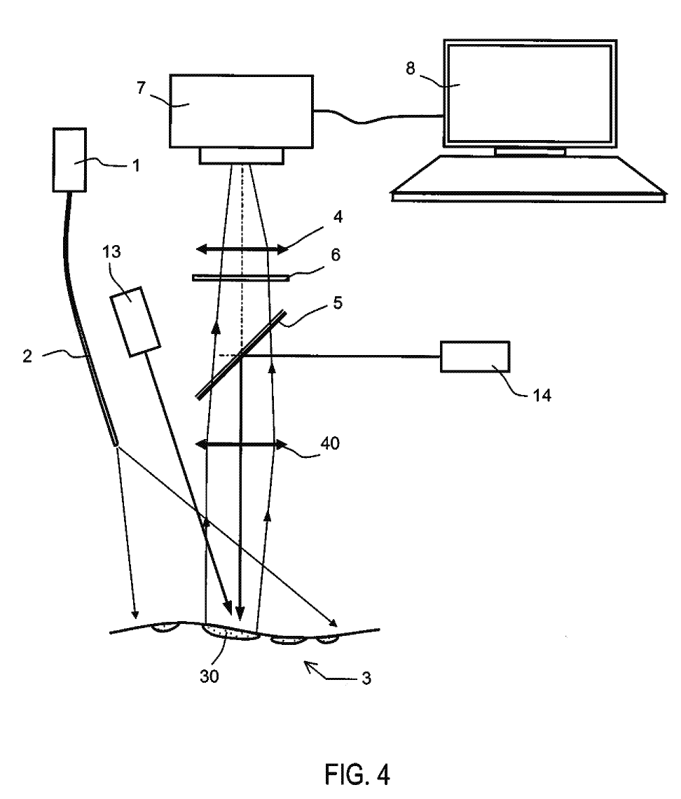

There are in principle two embodiments of probes according to the invention. In the first embodiment, one superimposes, on the biological tissue surface to be operated on, an image of the same area in visible light. In a simpler embodiment, one superimposes, on a marked area, a quasi-punctiform illumination in visible light, allowing the area to be easily located. It is of course possible to couple the two devices in such a way as to have both a complete illumination of the area to be operated on and also a more intense illumination of a particular area. As non-limiting examples, FIGS. 1 and 2 show two variants of the first embodiment, and FIGS. 3 and 4 show two variants of the second embodiment.

FIG. 1 is a schematic representation of a probe according to the invention, designed according to the first embodiment. It comprises essentially the elements described below.

A first light source 1 emits a first light beam intended to illuminate a biological tissue surface 3, of which at least one area 30, the marked area, has previously been fixed by a fluorescent marker. The techniques for marking living tissue are well known, moreover, and do not fall within the scope of this description. The beam must be emitted at a first wavelength corresponding substantially to the wavelength of excitation of the marking fluorophore. This effect can be obtained using various sources. Thus, the source can be a laser, a light-emitting diode or an incandescent lamp. It can comprise auxiliary optical devices for guiding or directing the light beam in order to improve its light distribution on the illuminated surface. Thus, by way of example, FIG. 1 shows a fibre optic 2 permitting transport of light from the source 1 to the illuminated area 3.

Under the effect of the excitation light, the fluorophore emits at a second wavelength. A first optical device of the camera type, comprising a first lens 4 and a photosensitive surface 7, forms a first image of the biological tissue surface 3 on the photosensitive surface 7. This photosensitive surface can be of the CCD type, for example. This first device can comprise a filter 6 whose transmission is adapted to this second wavelength. It is thus possible to avoid all stray light issuing either from the first source or from other light sources arranged in the operating theatre.

This first image can be processed by suitable image-processing means with which it is possible to reveal the marked areas present in the image. The simplest forms of processing are image processing involving intensity thresholding, but it is possible to employ more complex processing. Examples that may be mentioned are the Pro Plus or Analysis image-processing software systems.

This processed image is transmitted to a second optical device comprising at least:
An image projector 9 which, from the signal issuing from the processing means, generates a second image at at least a third wavelength situated in the visible spectrum, said second image being representative of the marked area. By way of example, as is illustrated in FIG. 1, the image projector used can be a display matrix functioning by transmission 9 illuminated by a source 10. This matrix can be of the liquid-crystal type. It is of course possible to use image projectors functioning by reflection, for example a micromirror matrix. It is also possible to add a filter 11 whose transmission is adapted to this third wavelength. This third wavelength can advantageously be situated in the green spectrum. This colour has the twin advantage of being situated in the area of greater sensitivity of the human eye and also of ensuring an excellent contrast with the red colour of the living tissues.
Of this second image, a second lens 12 forms a third image on the biological tissue surface 3, the second device being arranged in such a way that this third visible image is of the same dimensions as and in coincidence with the biological tissue surface.

Thus, the diseased areas are illuminated by a coloured light, facilitating the work of the surgeon who does not constantly need to look at a viewing screen.

The assembly composed of the first source and the first and second optical devices can be mounted in a common structure. This structure can be arranged in such a way as to be easily manoeuvrable by the operating surgeon. In this case, the probe has a field of the order of 8 centimetres, a working distance of the order of 10 to 15 centimetres, and a resolution of below 100 microns.

In the example in FIG. 1, the first optical device and the second device have separate optical axes. This arrangement has two drawbacks. On the one hand, the size of the probe is quite considerable. On the other hand, at least one of the optical axes shown in dot-and-dash lines in FIG. 1, of the first or second optical device, is not perpendicular to the mean plane of the biological tissue surface, which circumstance is not favourable to obtaining a good-quality image free of optical aberrations. It is thus possible to overcome these different drawbacks by interposition of a splitter plate, which will combine the fluorescent light issuing from the living tissues, which is at the second wavelength, with the light issuing from the image projector, which is at a third wavelength different from the second wavelength.

An embodiment of this type is illustrated in FIG. 2. This probe comprises the elements described below.

A first light source 1 emits a first light beam intended to illuminate a biological tissue surface 3 at a first wavelength. Under the effect of the excitation light, the fluorophore emits at a second wavelength.

A first optical device of the camera type, comprising a first lens composed of the optical assemblies 4 and 40, a splitter plate 5 and a photosensitive surface 7, forms a first image of the biological tissue surface 3 on the photosensitive surface 7. By way of example, the splitter plate can be a splitter cube or a dichroic plate. This first device can also comprise a filter 6 whose transmission is adapted to this second wavelength. In FIG. 2, the splitter plate is included between two optical assemblies 4 and 40 and functions by transmission. It is of course possible to adopt other configurations. For example, it is possible to use a single optical assembly arranged before or after the plate, and the dichroic plate can function on the camera path by transmission or reflection.

As in the case of FIG. 1, this first image can be processed by suitable image-processing means with which it is possible to reveal the marked areas present in the image.

This processed image is transmitted to a second optical device comprising at least:
An image projector 9 which, from the signal issuing from the processing means, generates a second image at at least a third wavelength situated in the visible spectrum, said second image being representative of the marked area. It is also possible to add a filter 11 whose transmission is adapted to this third wavelength.
A second lens 12 which, after this second image has been reflected on the splitter plate 5, forms a third image on the biological tissue surface 3, the second device being arranged in such a way that this third visible image is of the same dimensions as and in coincidence with the biological tissue surface, the parts of the optical axes of the first lens and of the second lens that are situated between the biological tissue surface and the splitter 5 being common.

When the surgeon moves the probe, the biological tissue surface changes. It is very important that the time between the moment the image is taken by the camera 7 and the moment it is presented by the display 9 is sufficiently short to ensure that the projected image has no perceptible offset with the surface of the living tissues.

A second embodiment of a probe according to the invention is depicted in FIGS. 3 and 4. The probe in FIG. 3 comprises essentially the elements described below:

A first light source 1 emitting a first light beam intended to illuminate a biological tissue surface 3 at a first wavelength. Under the effect of the excitation light, the fluorophore emits at a second wavelength.

A first optical device of the camera type, comprising a first lens 4 and a photosensitive surface 7, forms a first image of the biological tissue surface 3 on the photosensitive surface 7. As in the preceding embodiments, this first device can comprise a filter 6.

This first image can be processed by suitable image-processing means 8 with which it is possible to reveal the marked areas present in the image and to display them on a computer screen or a monitor.

A second optical device comprising at least a second light source 13 emitting a second beam at the first wavelength and arranged in such a way as to illuminate the centre of the biological tissue surface in a quasi-punctiform manner.

This second device can comprise a fourth light source 14 emitting a fourth beam at a fourth wavelength situated in the visible spectrum, and arranged in such a way as to illuminate the centre of the biological tissue surface in a quasi-punctiform manner in coincidence with the third beam. The third and fourth sources can be, for example, lasers or collimated laser diodes.

This second device has numerous advantages. On the one hand, by positioning the beams of the third and fourth sources at the centre of the field of the probe, one can be certain that the probe is at the working distance permitting optimal definition on the viewing screen. On the other hand, once the surgeon detects a marked area on the screen, he can activate the third and fourth sources and bring the detected area to the center of the field, where it is then optimally illuminated both in fluorescent light and also in visible light. This results in a good-quality image on the viewing screen, and an image that can be clearly seen by the operating surgeon.

An advantageous refinement of this device is that the image-processing means comprise means for identifying the marked areas, and that the probe comprises means for activating the fourth light source when a marked area is illuminated by the third light beam.

The probe depicted in FIG. 4 represents a variant of the preceding one. In this variant, as has already been described regarding the probe in FIG. 2, a splitter plate 5 is used which makes it possible to combine the optical beams issuing from the light sources 13 and 14.

It is possible to combine the devices described in FIGS. 1 and 2 with those described in FIGS. 3 and 4, this requiring only simple adaptations of the optical systems. A probe is then obtained which is able to project both a visible image, in coincidence with the biological tissue surface, and a spot of light illuminating the central area of the tissues. The different advantages of the preceding embodiments are in this way brought together in one and the same device.

The probe can be carried manually by the operating surgeon or can be carried by a telescopic arm. The probe is part of a medical apparatus that generally comprises its own sources of ambient lighting. Lighting of the scialytic type is generally used for this purpose. It is in this case expedient to adapt these sources of lighting in such a way that their wavelengths are if possible different from at least the first wavelength and the second wavelength which corresponds to the wavelength of fluorescence of the marked areas. This is necessary to avoid a situation where the component of the light emitted by the scialytic lighting, corresponding to the fluorescence wavelength, is reflected by the patient and appears on the first image formed on the photosensitive surface 7.

It will be readily seen by one of ordinary skill in the art that the present invention fulfils all of the objects set forth above. After reading the foregoing specification, one of ordinary skill in the art will be able to affect various changes, substitutions of equivalents and various aspects of the invention as broadly disclosed herein. It is therefore intended that the protection granted hereon be limited only by definition contained in the appended claims and equivalents thereof.

1. A peroperative optical probe comprising:
a first light source emitting a first light beam intended to illuminate a biological tissue surface, of which at least one area, marked area, has previously been fixed by a fluorescent marker, said beam being emitted at a first wavelength corresponding substantially to the wavelength of excitation of the marking fluorophore, said fluorophore emitting at a second wavelength;
a first optical device comprising a first lens and a photosensitive surface, said lens forming, on said photosensitive surface, a first image of the biological tissue surface, the first device being connected to means for processing the image issuing from the photosensitive surface;
optical means that are different from the first optical device and that are designed to illuminate at least a part of the biological tissue surface, the surface part illuminated by the optical means preferably corresponding to at least one of the marked zones identified by the means for processing the image.

2. The peroperative optical probe according to claim 1, wherein the optical means comprise at least one image projector which, at the command of a control device analyzing the signal issuing from the processing means, generates a second image at at least a third wavelength situated in the visible spectrum, said second image being representative of the marked area, of which second image a second lens forms a third image on the biological tissue surface, the second device being arranged in such a way that this third visible image is of the same dimensions as and in coincidence with at least a marked area of the biological tissue surface.

3. The peroperative optical probe according to claim 2, wherein the optical means comprise a splitter allowing the second wavelength to be divided spectrally from the third wavelength, said splitter being arranged in such a way that the parts of the optical axes of the first lens and of the second lens that are situated between the biological tissue surface and said splitter are common.

4. The peroperative optical probe according to claim 1, wherein the first optical device comprises a first optical filter transparent to the second wavelength.

5. The peroperative optical probe according to claim 2, wherein the image projector comprises a second optical filter transparent to the third wavelength.

6. The peroperative optical probe according to claim 1, wherein the optical means comprise at least a third light source emitting a third beam at the first wavelength and arranged in such a way as to illuminate the centre of the biological tissue surface in a quasi-punctiform manner.

7. The peroperative optical probe according to claim 6, wherein the optical means comprise a fourth light source emitting a fourth beam at a fourth wavelength situated in the visible spectrum, and arranged in such a way as to illuminate the centre of the biological tissue surface in a quasi-punctiform manner in coincidence with the third beam.

8. The peroperative optical probe according to claim 7, wherein the optical means comprise a splitter allowing the second wavelength to be divided spectrally from the fourth wavelength, said splitter being arranged in such a way that the parts of the optical axes of the first lens and of the fourth source that are situated between the biological tissue surface and said splitter are common.

9. The peroperative optical probe according to claim 6, wherein the optical means comprise an image generator which, from the signal issuing from the processing means, generates a second image at at least a third wavelength situated in the visible spectrum, said second image being representative of the marked area, of which second image a second lens forms a third image on the biological tissue surface, the optical means being arranged in such a way that this third visible image is of the same dimensions as and in coincidence with the biological tissue surface.

10. The peroperative optical probe according to claim 7, wherein the image-processing means comprise means for identifying the marked areas, and in that said probe comprises electronic means for activating the fourth light source when a marked area is illuminated by the third light beam.

11. Medical apparatus designed to be connected to a probe according to claim 7, comprising said processing means and the device for control of the fourth light source.

12. Medical apparatus designed to be connected to a probe according to claim 2, comprising a display device for viewing the equivalent of the first image or of the second image.

13. Medical apparatus designed to be connected to a probe according to claim 1, comprising lighting of the scialytic type that emits at wavelengths different from at least the first wavelength.

14. The peroperative optical probe according to claim 7, wherein the optical means comprise an image generator which, from the signal issuing from the processing means, generates a second image at at least a third wavelength situated in the visible spectrum, said second image being representative of the marked area, of which second image a second lens forms a third image on the biological tissue surface, the optical means being arranged in such a way that this third visible image is of the same dimensions as and in coincidence with the biological tissue surface.

15. The peroperative optical probe according to claim 8, wherein the optical means comprise an image generator which, from the signal issuing from the processing means, generates a second image at at least a third wavelength situated in the visible spectrum, said second image being representative of the marked area, of which second image a second lens forms a third image on the biological tissue surface, the optical means being arranged in such a way that this third visible image is of the same dimensions as and in coincidence with the biological tissue surface.
